(19) United States
(12) Patent Application Publication
(10) Pub. No.: US 2022/0183800 A1
(43) Pub. Date: Jun. 16, 2022

(54) SCAN BODIES FOR DIGITAL IMPRESSIONS AND SYSTEMS, INDICIA, AND METHODS OF USING SCAN BODIES

(71) Applicant: Milan Madhavji, Mississauga (CA)

(72) Inventor: Milan Madhavji, Mississauga (CA)

(21) Appl. No.: 17/543,131

(22) Filed: Dec. 6, 2021

Related U.S. Application Data
(60) Provisional application No. 63/123,794, filed on Dec. 10, 2020.

(51) Int. Cl. A61C 9/00; A61B 90/00

(52) U.S. Cl. CPC A61C 9/0053; A61B 2090/3937; A61B 2090/3912; A61B 90/39

(57) ABSTRACT

A scan body member includes a body portion having a first end portion and second end portion opposite the first end portion. A wall portion extends between the first end portion and the second end portion. The wall portion has an exterior surface portion. A system of indicia has a non-recurring distribution about the exterior surface portion and is recessed within the wall portion. The system of indicia is scannable by an optical scanning system to provide unique scan body imaging information for constructing computerized models.

### CROSS REFERENCE TO RELATED APPLICATIONS

This application claims the benefit of U.S. patent application Ser. No. 63/123,794 filed Dec. 10, 2020, whose contents are incorporated herein by reference.

### TECHNICAL FIELD

The present disclosure generally relates to non-recurring or unique, non-repeating surface textures on intra-oral scan bodies, as well as the usage of multiple uniquely textured intra-oral scan bodies during acquisition of digital impressions of an intra-oral cavity.

### BACKGROUND

Dental implants are used in restorative dentistry to affix a dental prosthesis to the dental arches of a patient, such as the mandible or maxilla. A model of the intra-oral structure of a patient is used for designing the dental prosthesis or prosthetics to be affixed within the intra-oral cavity of the patient at implant sites.

Digital impressions are a technology which allows the creation of virtual, computer-generated model of the hard and soft tissues of the oral cavity of a patient. Digital impressions are generated from three-dimensional (3D) topographic image data acquired from the oral cavity of the patient using optical scanning devices.

Planning the position, height, angle and orientation of the prosthetic within the mouth of a patient includes digital modeling of scan bodies engaged in the implant site. Digital impressions of the intra-oral cavity with the scan bodies mounted within the mouth of the patient relay the position, height, angle and orientation information for planning and positioning of a physical dental implant.

In order to generate a digital impression with the scan bodies engaged, the 3D topographic image data is combined or “stitched” together to create the 3D surface model of the hard and soft tissues of the oral cavity, including the dental condition of the patient. In the case of digital impressions of intra-oral scan bodies, this reliance on matching of 3D contours to produce a stitched final result is predicated on the concept of the surfaces being sufficiently unique that they can be reliably matched and stitched together.

Scan bodies are typically cylindrical or nearly cylindrical in shape and do not typically include any indicia for mapping intra-oral structures for positioning relative to the scan body. Scan bodies are generally very uniform in shape and appearance and are often mis-stitched due to incorrect matching of stitching data when multiple matches can be made between the uniform surface to be stitched and the existing data. For example, an arc-shaped object would be able to be matched to a cylindrical surface many ways because the cylindrical shape of the scan body is generally uniform around an entire axis thereof.

It therefore desirable to have a means for reducing or potentially eliminating mis-stitching errors between the surface to be stitched and the 3D topographic data when creating a 3D surface model.

### SUMMARY

The present disclosure generally relates to non-recurring or unique, non-repeating surface textures on intra-oral scan bodies, as well as the usage of multiple uniquely textured intra-oral scan bodies during acquisition of digital impressions of an intra-oral cavity.

In one aspect of the invention, there is provided a scan body member including a body portion having a first end portion and a second end portion opposite the first end portion. A wall portion extends between the first end portion and the second end portion. The wall portion has an exterior surface portion. The scan body member further includes a system of indicia having a non-recurring distribution about the exterior surface portion and recessed within the wall portion. The system of indicia may be scannable by an optical scanning system.

The scan body member may include an oblique wall portion extending from the second end portion and intersecting the wall portion between the second end portion and the first end portion. The system of indicia may be distributed about a surface portion of the oblique wall portion.

The system of indicia may include a plurality of circular depressions extending into the wall portion from the exterior surface portion. In another aspect, the system of indicia may include at least one valley extending at least partially about the wall portion, the valley extending into the wall portion from the exterior surface portion. The at least one valley may vary in depth therealong. The system of indicia may include a plurality of valleys and a plurality of ridges between the valleys. The system of indicia may vary in depth of recess within the wall portion. Variation in depth may be according to a depth gradient.

In another aspect, there is provided a system for generating digital impressions. At least one scan body member has a body portion having a first end portion and a second end portion opposite the first end portion. A wall portion may extend between the first end portion and the second end portion, the wall portion having an exterior surface portion. The system may include a system of indicia having a non-recurring distribution about the exterior surface portion and recessed within the wall portion and at least one optical scanning device for scanning the at least one scan body member with the system of indicia.

The at least one scan body member may include at least one oblique wall portion extending from the second end portion and intersecting the wall portion between the second end portion and the first end portion.

The system of indicia may include a plurality of circular depressions extending into the wall portion from the exterior surface portion. In another aspect, the system of indicia may include at least one valley extending at least partially about the wall portion, the valley extending into the wall portion from the exterior surface portion. The system of indicia may include a plurality of valleys and a plurality of ridges between the valleys.

In another aspect, the at least one scan body member includes a plurality of scan body members and each scan body member of the plurality of scan body members has a unique system of indicia.

The system of indicia may vary in depth of recess within the wall portion. Variation in depth may be according to a depth gradient.

In another aspect, there is provided a method for generating a digital model of an intra-oral cavity comprising the steps of: providing at least one scan body member, each scan body member comprising: a body portion having a first end portion and a second end portion opposite the first end portion; a wall portion extending between the first end portion and the second end portion, the wall portion having an exterior surface portion; and, a system of indicia having a non-recurring distribution about the exterior surface portion and recessed within the wall portion, affixing, within an intra-oral cavity, the at least one scan body member, scanning the at least one scan body member and system of indicia with an optical scanning device to provide image data representing the intra-oral cavity and, generating a model of the intra-oral cavity based on the image data representing the intra-oral cavity.

The step of generating a model may further include the steps of determining the position of the image data in the model relative to the scanned system of indicia of the at least one scan body member, and positioning the image data in the model.

The step of providing at least one scan body member may further include providing a plurality of scan body members, and each scan body member of the plurality of scan body members has a unique system of indicia.

In another aspect, there is provided a system of indicia including a plurality of recessed indicia having a non-recurring distribution about an exterior surface portion of a wall portion of a scan body member and extending into the wall portion. The scan body member may have a body portion having a first end portion and a second end portion. The wall portion may extend between the first end portion and the second end portion. The plurality of recessed indicia may be scannable by an optical scanning system.

The plurality of recessed indicia may be a plurality of circular depressions. In another aspect, the plurality of recessed indicia may include a plurality of valleys each extending at least partially about the wall portion. The plurality of recessed indicia may vary in depth of recess within the wall portion. Variation in depth may be according to a depth gradient.

The system of indicia provides each scan body with a uniquely textured surface or profile. The surface texture provides unique information which is detectable by an oral imaging device. When positioned within an oral cavity, the unique surface texture information of the scan bodies may be used to facilitate modeling of the oral cavity.

### BRIEF DESCRIPTION OF THE DRAWINGS

Exemplary non-limiting embodiments are described with reference to the accompanying drawings in which:

FIG. 1 is a perspective view of a scan body according to one aspect of the present invention;

FIG. 2 is a view of the scan body of FIG. 1 with an illustration of the recess mapping applied to the scan body;

FIG. 3 is a perspective view of a scan body according to another aspect the present invention;

FIG. 4 is a second perspective view of the scan body of FIG. 3;

FIG. 5 is an illustrative representation of a three-dimensional model of an intra-oral cavity with four scan bodies affixed to a dental arch;

FIG. 6 is a perspective view of a conventional scan body;

FIG. 7 is a perspective view of a scan body according to the present invention juxtaposed with a conventional scan body;

FIG. 8 is a second perspective view of the scan body according to the present invention juxtaposed with the conventional scan body of FIG. 7; and,

FIG. 9 is a top-down perspective view of the scan body according to the present invention juxtaposed with the conventional scan body of FIG. 7.

### DESCRIPTION

The present disclosure generally relates to non-recurring or unique, non-repeating surface textures on intra-oral scan bodies, as well as the usage of multiple uniquely textured intra-oral scan bodies during acquisition of digital impressions of an intra-oral cavity.

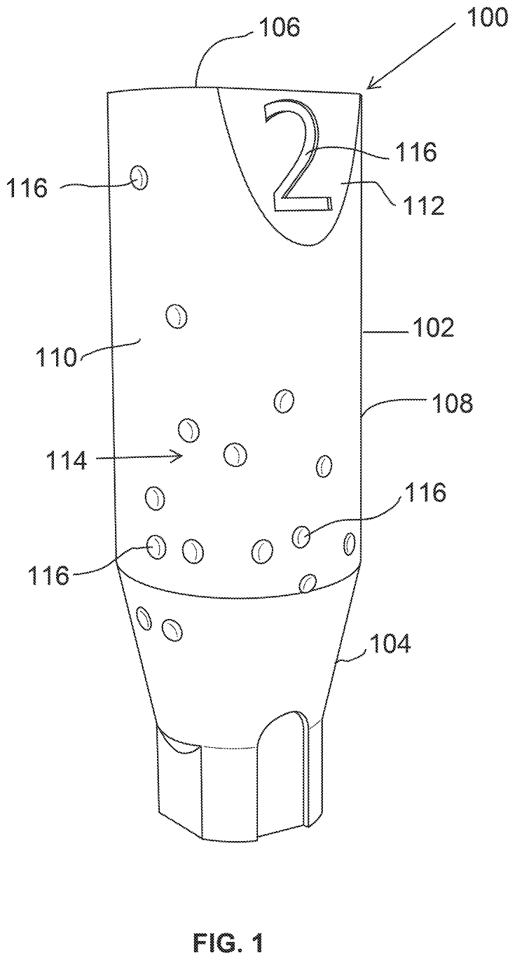

In FIG. 1, there is shown an intra-oral scan body or scan body member 100 according to one aspect of the present invention. The scan body 100 includes a body portion 102 having a first end portion 104 and a second end portion 106 opposite the first end portion 104. The scan body 100 may be affixed, via the first end portion 104, to a dental arch, such as the maxilla (not shown) and/or mandible (not shown) of a patient at a dental implant site (not shown) passing through the gingiva (not shown). A wall portion 108 extends between the first end portion 104 and the second end portion 106. The wall portion 108 has an exterior surface portion 110. The scan body 100 may include an asymmetric feature, such as oblique wall portion 112 to provide directional information of the scan body 100.

In FIG. 1, the asymmetric feature is shown as being oblique wall portion 112 extending from the second end portion 106 and intersecting the wall portion 108 between the second end portion 106 and the first end portion 104. However, the asymmetric feature may take other forms suitable for informing a user as to the orientation of the scan body and which is detectable by an optical scanning system.

Scan body 100 further includes a system of indicia 114 having a non-recurring distribution about the exterior surface portion 110. Preferably, the indicia are recessed within the wall portion 108. The indicia may, for example, be provided by engraving, molding, additive manufacturing or any other suitable means for forming the indicia. Thereby, the indicia 114 contribute to a texturization of the exterior surface portion 110 of the scan body 100. The system of indicia 114 is scannable by an optical scanning system. A suitable optical scanning system is capable of detecting the depth of the indicia 114 because they are recessed into the wall portion 108.

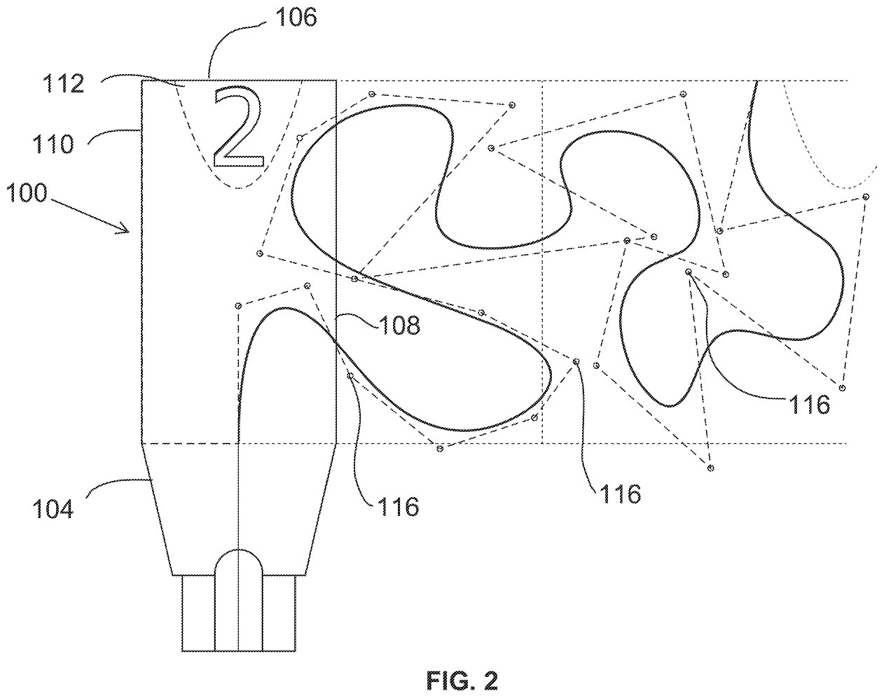

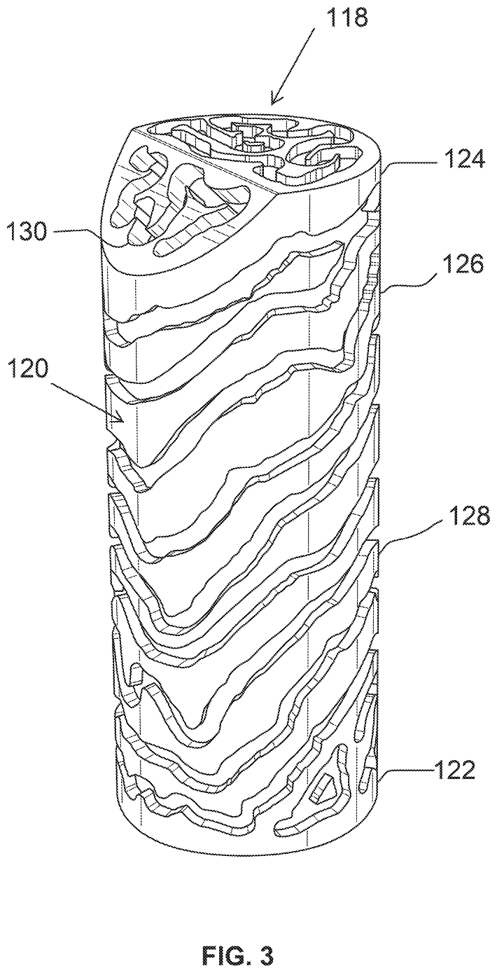

The indicia 116 in the system of indicia 114 may take any suitable form. As shown in FIG. 1, the indicia 116 are circular depressions extending into the wall portion 108 from the exterior surface portion 110. At least one of the wall portion 108 and the oblique wall portion 112 may further include an indicia 116 in the form of a number, such as a “2” recessed therein. In FIGS. 1 and 2, the number “2” is positioned on the oblique wall portion 112. In another aspect, the indicia 116 may include one or more valleys extending at least partially about the wall portion 108, with each valley extending into the wall portion 108 from the exterior surface portion 110. Preferably, the system of indicia 114 includes a plurality of valleys and a plurality of ridges between the valleys. Such an arrangement could be used to produce a fingerprint-type of system of indicia 114 in a computer-generated model, as shown in more detail in FIG. 3. It should be understood that the indicia 116 may take any suitable form and that the system of indicia 114 may include indicia 116 of different types, such as a combination of circular depressions and valleys, for example.

The indicia 116 may be further distinguishable from one another by applying a depth gradient. For example, an indicia 116 which consists of an elongate valley, such as that modeled in FIG. 3, may vary in depth along the length thereof. This may impart information pertaining to the direction or orientation of the scan body. Moreover, depth of recess may vary from one indicia 116 to the next. Accordingly, individual indicia 116, the system of indicia 114 or members or groups of indicia in the system of indicia 116 may be recessed into the wall portion 108 according to different or varying depths.

FIG. 2 is a two-dimensional perspective view of the distribution or layout of the indicia 116 in the system of indicia 114 about the wall portion 108 of the scan body 100 shown in FIG. 1. The distribution of the indicia 114 is non-repeating, or unique about the wall portion 108. Thereby, regions of the scan body 100 may be distinguished from one another based on the relative position of the indicia 116 in that region. Moreover, the system of indicia 114 is preferably distinct from one scan body 100 to the next. In instances where multiple scan bodies 100 are positioned within an intra-oral cavity, the scan bodies 100 would be distinguishable from one another.

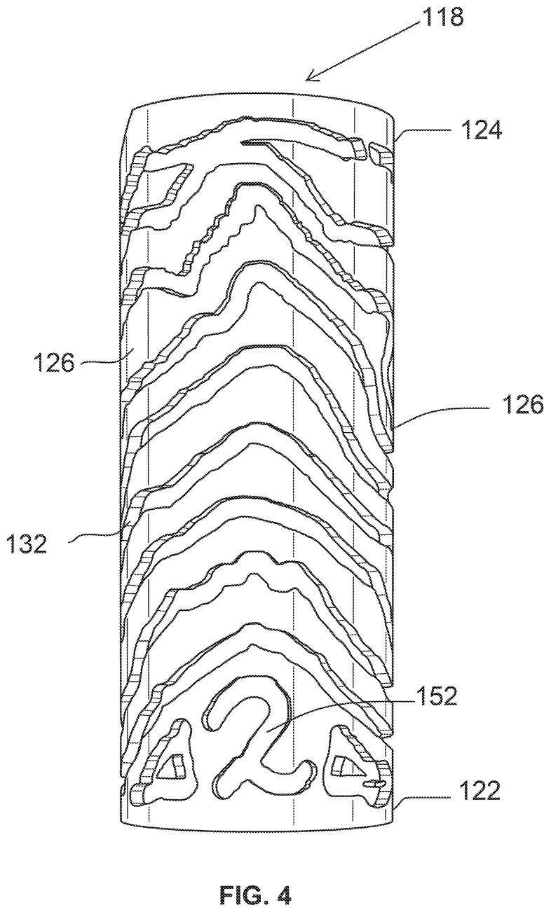

A model 118 of a scan body 100 according to one aspect of the present invention is shown in FIGS. 3 and 4. The modeled scan body 118 includes a modeled body portion 120 having a first end portion 122 and a second end portion 124 opposite the first end portion 122. A modeled wall portion 126 extends between the first end portion 122 and the second end portion 124. The modeled wall portion 126 has an exterior surface portion 128. The modeled scan body 118 may include a modeled oblique wall portion 130.

The model 118 of scan body 100 further includes the modeled system of indicia 132. As with the system of indicia 114 of a physical scan body 100, the modeled system of indicia 132 is represented as non-recurring, or as non-repeating, about the exterior surface portion 128. The modeled system of indicia 132 corresponds with the form of the system of indicia 114 on the physical scan body from which the image data was obtained. In the aspect shown in FIG. 3, the system of indicia 114 scanned by the optical scanning device was of a fingerprint-type system of indicia 114 applied to the wall portion of the scan body and the oblique wall portion. Therefore, the modeled system of indicia 132 is of a fingerprint-type on the modeled wall portion 126 and the modeled oblique wall portion 130.

It should be understood that if the system of indicia 114 were of the circular depression type shown in FIGS. 1 and 2, then the modeled system of indicia 132 would be correspondingly represented as modeled circular depressions.

When modeling an intra-oral cavity containing one or more scan bodies 100 according to the present invention, the optical scanning device will obtain image information from the system of indicia 114 of each scan body 100 positioned within the intra-oral cavity. The unique information provided by the unique system of indicia of each scan body 100 may be used to stitch the image data more precisely to the correct position in the model of the intra-oral cavity.

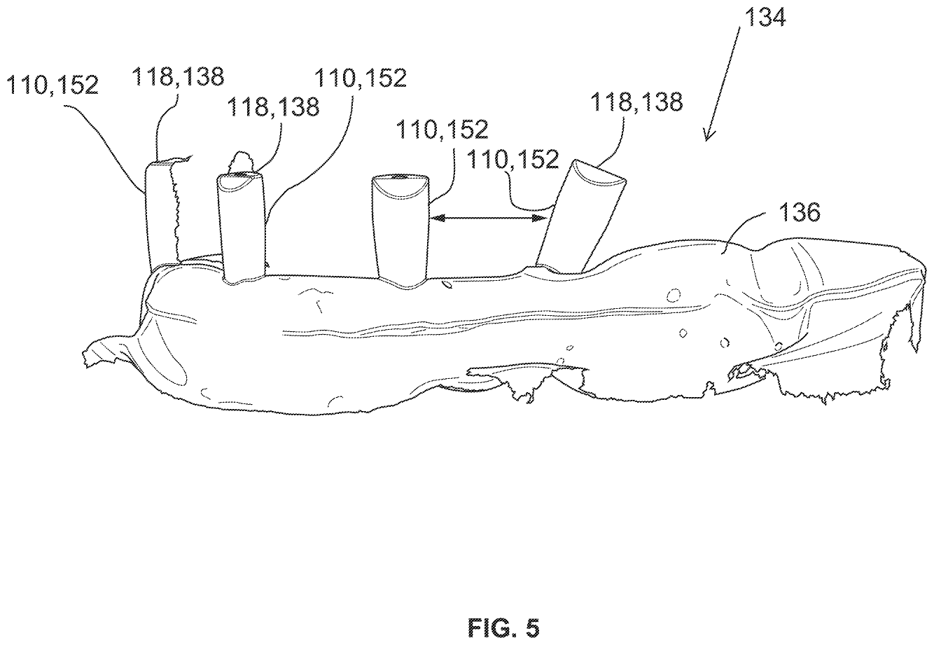

A three-dimensional computer-generated model 134 of an intra-oral cavity is shown in FIG. 5. The model 134 includes representations of the gingiva 136 and representations of intra-oral scan bodies, which would have been affixed to the maxilla (not shown) and/or mandible (not shown) of a patient at implant sites (not shown) passing through the gingiva (not shown) prior to generation of the model 134. The representations of the intra-oral scan bodies may be modeled scan bodies 118 according to aspects of the present invention or modeled conventional scan bodies 138 to be discussed further hereinafter. Where scan bodies according to aspects of the present invention are used, the exterior surface portions 110 will include the modeled system of indicia 132. Where conventional scan bodies are used, which do not have a system of indicia 116, the modeled conventional exterior surface portions 148 will be smooth.

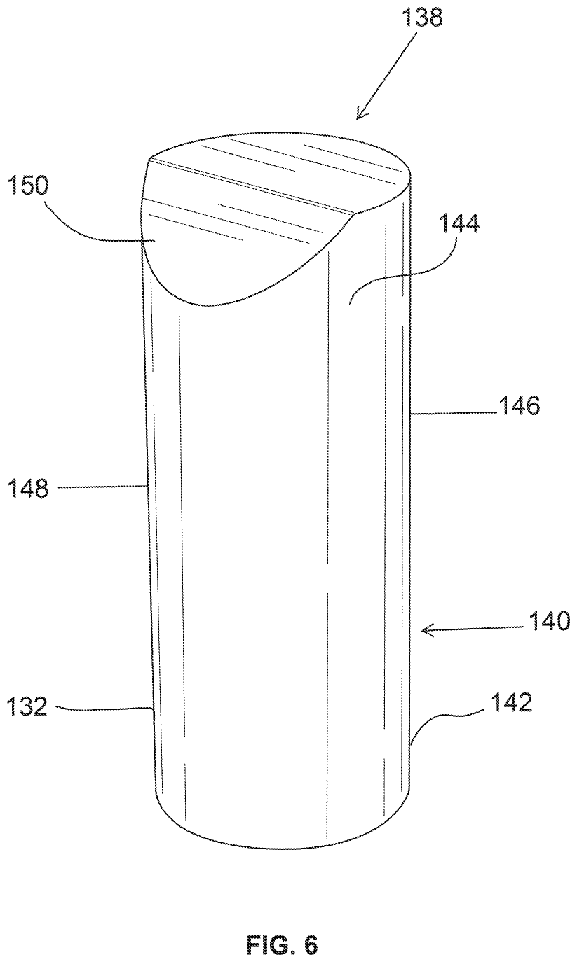

A modeled conventional scan body 138 is shown in greater detail in FIG. 6. The modeled conventional scan body 138 includes a modeled body portion 140 having a first end portion 142 and a second end portion 144 opposite the first end portion 142. A modeled wall portion 146 extends between the first end portion 142 and the second end portion 144. The modeled wall portion 146 has an exterior surface portion 148. The modeled scan body 138 may include a modeled oblique wall portion 150.

The model of the intra-oral cavity shown in FIG. 5 is constructed from data provided by multiple intra-oral scans using suitable optical scanning systems. The data from different scans is combined or “stitched” together in order to construct a finished model. However, as shown in FIG. 6, the exterior surface portion 148 of the modeled conventional scan body 138 does not include indicia which would distinguish one region of the scan body 138 from another region thereof or one conventional scan body 138 from another conventional scan body 138 from the same set of intra-oral scans.

Due to the general uniformity of the exterior surface portion 148 of the modeled conventional scan body 138, any image data obtained from intra-oral scans which does not include the surrounding gingival regions is difficult to stitch. Models of the gingival regions include unique information which may be considered when stitching the model. However, this does not provide any unique information that would assist with relative positioning of image information relating to the scan bodies. Therefore, accurate stitching of image data to complete the model 134 using conventional scan bodies 138 is very challenging.

Typically, the maximum number of implants per dental arch is limited. Currently, a dental arch may accommodate approximately eight implants. So, a set of scan bodies according to this aspect of the invention would contain at least 8 variants for each size and type of scan body, for example. Each unique scan body may be numbered, from 1 to 8, for example, or otherwise identified so that they can be visually distinguishable from one another by a human user. For example, a user affixing the physical scan bodies would know not to use two scan bodies labeled with the number “2”. If the user had to scan 8 implants, they would use 8 different scan bodies. In FIG. 4, there is shown one aspect wherein the modeled system of indicia 132 includes a representation of the number “2” 152. Accordingly, the modeled scan bodies 118 can further include the numbered indicia 152 which may further facilitate the stitching process.

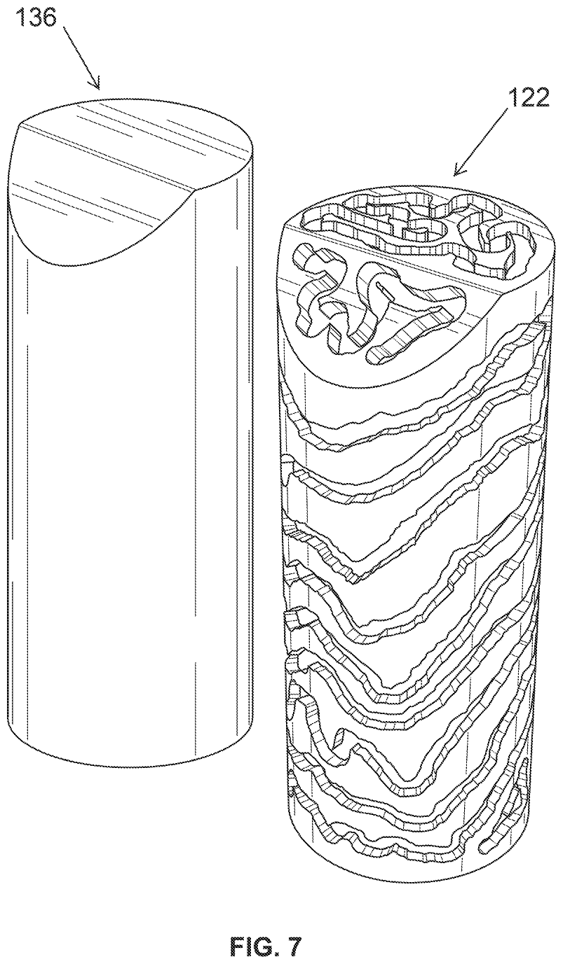

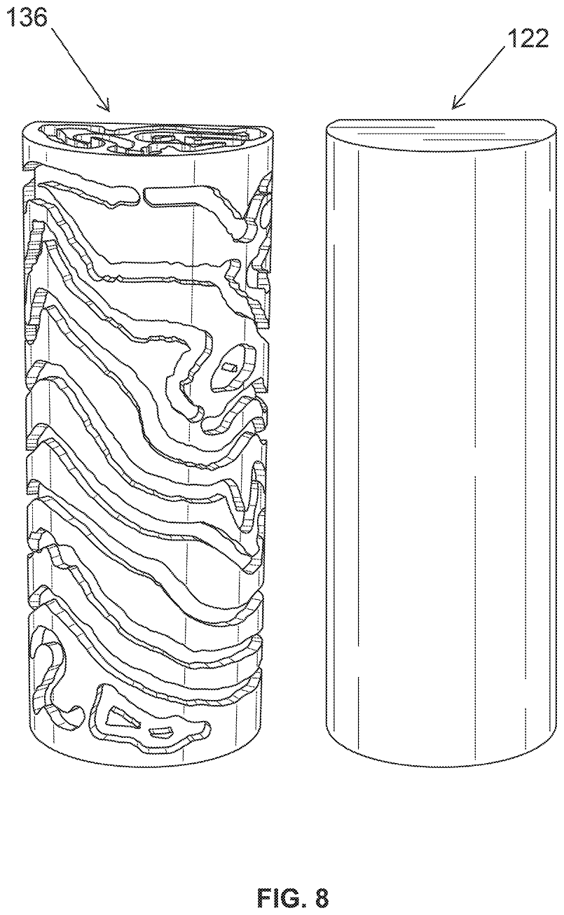

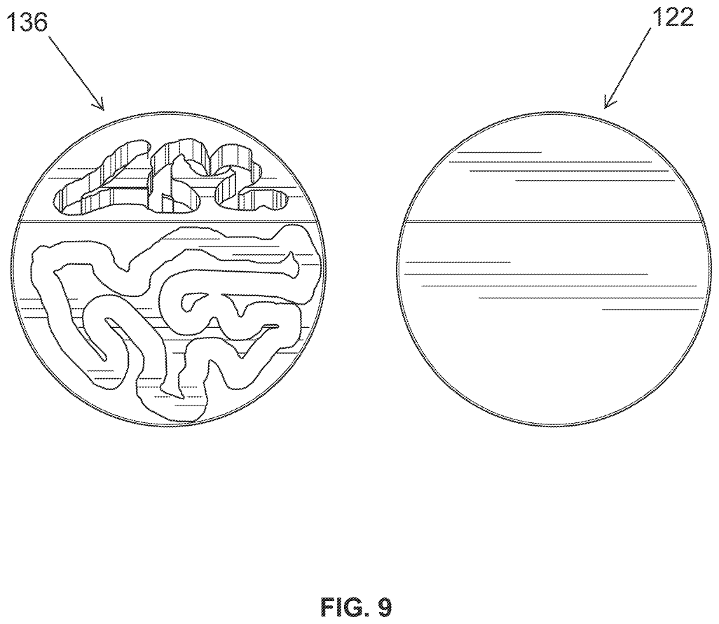

In FIGS. 7, 8 and 9 there is shown a modeled scan body 118 according to the aspect shown in FIG. 3 juxtaposed with a modeled conventional scan body 138 shown in FIG. 6, for comparison purposes. The modeled system of indicia 132 of the modeled scan body 118 provides distinct elements which are not present on the modeled conventional scan body 138. The uniformity of the modeled wall portion in the modeled scan body 118 is uniquely disrupted such that no surfaces exhibit the same topography. This reduces or eliminates the potential for errors in the stitching operation when creating a 3D model from the topographic image data of the intra-oral cavity retrieved during the optical scanning operation. Thereby, there is provided an expedient reliable means for modeling an intra-oral cavity. A digital impression system utilizing the system of indica 114 on the intra-oral scan bodies 100 as described above would likewise provide an efficient means for modeling an intra-oral cavity.

Moreover, unique topography may be applied by retrofit to any scan body by any manufacturer. For example, a conventional scan body could be engraved with a system of indicia in order to provide the advantages described herein to convention scan bodies. Further, the application of the system of indicia may be made to conventional scan bodies in a manner which permits backward compatibility with the modeling systems of manufacturers while still permitting high quality scanning.

While the invention has been described in terms of specific aspects, it is apparent that other forms could be adopted by one skilled in the art. For example, the methods described herein could be performed in a manner which differs from the aspects described herein. The steps of each method could be performed using similar steps or steps producing the same result but which are not necessarily equivalent to the steps described herein. Some steps may also be performed in different order to obtain the same result. Similarly, the apparatuses and systems described herein could differ in appearance and construction from the aspects described herein, the functions of each component of the apparatus could be performed by components of different construction but capable of a similar though not necessarily equivalent function, and appropriate materials could be substituted for those noted. Accordingly, it should be understood that the invention is not limited to the specific aspects described herein. It should also be understood that the phraseology and terminology employed above are for the purpose of disclosing the illustrated aspects, and do not necessarily serve as limitations to the scope of the invention.

What is claimed is:

1. A scan body member comprising:
a body portion having a first end portion and a second end portion opposite the first end portion;
a wall portion extending between the first end portion and the second end portion, the wall portion having an exterior surface portion; and
a system of indicia having a non-recurring distribution about the exterior surface portion and recessed within the wall portion.

2. The scan body member according to claim 1, wherein the system of indicia being scannable by an optical scanning system.

3. The scan body member according to claim 1, wherein the scan body member includes an oblique wall portion extending from the second end portion and intersecting the wall portion between the second end portion and the first end portion.

4. The scan body member according to claim 3, wherein the system of indicia is distributed about a surface portion of the oblique wall portion.

5. The scan body member according to claim 1, wherein the system of indicia includes a plurality of circular depressions extending into the wall portion from the exterior surface portion.

6. The scan body member according to claim 1, wherein the system of indicia includes at least one valley extending at least partially about the wall portion, the valley extending into the wall portion from the exterior surface portion.

7. The scan body member according to claim 1, wherein the system of indicia includes a plurality of valleys and a plurality of ridges between the valleys.

8. The scan body member according to claim 1, wherein the system of indicia varies in depth of recess within the wall portion.

9. The scan body member according to claim 6 wherein the at least one valley varies in depth therealong.

10. The scan body member according to claim 9, wherein variation in depth is according to a depth gradient.

11. A system for generating digital impressions, the system comprising:
at least one scan body member having a body portion having a first end portion and a second end portion opposite the first end portion;
a wall portion extending between the first end portion and the second end portion, the wall portion having an exterior surface portion;
a system of indicia having a non-recurring distribution about the exterior surface portion and recessed within the wall portion; and
at least one optical scanning device for scanning the at least one scan body member with the system of indicia.

12. The system according to claim 11, wherein the at least one scan body member includes at least one oblique wall portion extending from the second end portion and intersecting the wall portion between the second end portion and the first end portion.

13. The system according to claim 11, wherein the system of indicia includes a plurality of circular depressions extending into the wall portion from the exterior surface portion.

14. The system according to claim 11, wherein the system of indicia includes at least one valley extending at least partially about the wall portion, the valley extending into the wall portion from the exterior surface portion.

15. The system according to claim 11, wherein the system of indicia includes a plurality of valleys and a plurality of ridges between the valleys.

16. The system according to claim 11, wherein the at least one scan body member includes a plurality of scan body members and each scan body member of the plurality of scan body members has a unique system of indicia.

17. The system according to claim 11, wherein the system of indicia varies in depth of recess within the wall portion.

18. The system according to claim 14 wherein the at least one valley varies in depth therealong.

19. The system according to claim 18, wherein variation in depth is according to a depth gradient.

20. A method for generating a digital model of an intra-oral cavity the method comprising the steps of:
providing at least one scan body member, each scan body member comprising:
a body portion having a first end portion and a second end portion opposite the first end portion;
a wall portion extending between the first end portion and the second end portion, the wall portion having an exterior surface portion;
a system of indicia having a non-recurring distribution about the exterior surface portion and recessed within the wall portion;
affixing, within an intra-oral cavity, the at least one scan body member;
scanning the at least one scan body member and system of indicia with an optical scanning device to provide image data representing the intra-oral cavity; and
generating a model of the intra-oral cavity based on the image data representing the intra-oral cavity.

21. The method according to claim 20, wherein the step of generating a model further comprises the steps of:
determining the position of the image data in the model relative to the scanned system of indicia of the at least one scan body member; and,
positioning the image data in the model.

22. The method according to claim 21, wherein the step of providing at least one scan body member further includes providing a plurality of scan body members, and each scan body member of the plurality of scan body members has a unique system of indicia.

23. A system of indicia comprising:
a plurality of recessed indicia having a non-recurring distribution about an exterior surface portion of a wall portion of a scan body member and extending into the wall portion, the scan body member having a body portion having a first end portion and a second end portion, the wall portion extending between the first end portion and the second end portion.

24. A system of indicia according to claim 23, wherein the plurality of recessed indicia are scannable by an optical scanning system.

25. The system of indicia according to claim 23, wherein the plurality of recessed indicia are a plurality of circular depressions.

26. The system of indicia according to claim 23, wherein the plurality of recessed indicia include a plurality of valleys each extending at least partially about the wall portion.

27. The system of indicia according to claim 23, wherein the plurality of recessed indicia vary in depth of recess within the wall portion.

28. The system of indicia according to claim 26 wherein each valley of the plurality of valleys varies in depth therealong.

29. The system of indicia according to claim 28, wherein variation in depth is according to a depth gradient.
